(19) United States
(12) Patent Application Publication
(10) Pub. No.: US 2020/0357525 A1
(43) Pub. Date: Nov. 12, 2020

(54) MEDICAL INFORMATION-PROCESSING SYSTEM

(71) Applicant: CANON MEDICAL SYSTEMS CORPORATION, Otawara-shi (JP)

(72) Inventors: Yusuke KANO, Nasushiobara (JP); Kazuki Utsunomiya, Nasushiobara (JP)

(73) Assignee: CANON MEDICAL SYSTEMS CORPORATION, Otawara-shi (JP)

(21) Appl. No.: 16/860,109

(22) Filed: Apr. 28, 2020

(30) Foreign Application Priority Data
May 9, 2019 (JP) 2019-089051

(51) Int. Cl. G16H 70/20; G06T 13/80; G06T 11/20; G06T 3/40; G16H 30/20; G16H 10/60

(52) U.S. Cl. CPC G16H 70/20; G06T 13/80; G16H 10/60; G06T 3/40; G16H 30/20; G06T 11/206

(57) ABSTRACT

A medical information-processing system according to an embodiment includes a processing circuitry. The processing circuitry displays medical data in a display region provided in a screen, and performs, according to an operation with respect to the medical data, processing of changing a display range of the medical data while displaying an animation that shows a process of changing the display range of the medical data in the display region, or processing of displaying another medical data related to the medical data subject to the operation in a highlighted manner.

### CROSS-REFERENCE TO RELATED APPLICATIONS

This application is based upon and claims the benefit of priority from Japanese Patent Application No. 2019-089051, filed on May 9, 2019; the entire contents of which are incorporated herein by reference.

### FIELD

Embodiments described herein relate generally to a medical information processing system.

### BACKGROUND

Medical practitioners, such as a doctor, have been practicing diagnosis and treatment of a subject (for example, patient), using various kinds of medical data comprehensively. Moreover, discussion on a treatment plan and the like of the subject by plural medical practitioners has also been practiced. For example, in a medical conference, determination of diagnosis and a treatment plan by conferring with plural medical practitioners while referring to medical information displayed on a screen has been practiced.

When a treatment plan is decided by plural medical practitioners as the above conference, for example, an attending doctor of a subject prepares materials to explain a condition based on medical data of the subject. In such a case, it is common for a medical practitioner that prepares materials to make static materials in which a particular concerned portion is enlarged or extracted. However, only by presenting static materials, it is difficult to grasp conditions of periphery of the particular portion or relations with other medical data, and is not convenient.

### BRIEF DESCRIPTION OF THE DRAWINGS

FIG. 1 illustrates a configuration example of a medical information-processing system according to an embodiment;

FIG. 2 illustrates an example of a configuration of the medical information-processing apparatus according to the embodiment;

FIG. 3 illustrates an example of a screen displayed by a display control function of the embodiment;

FIG. 4 illustrates an example of a screen displayed by the display control function of the embodiment;

FIG. 5 is an illustration for explaining an example of actions of a display supporting function according to the embodiment;

FIG. 6 is an illustration for explaining an example of actions of the display supporting function according to the embodiment;

FIG. 7 is an illustration for explaining an example of actions of the display supporting function according to the embodiment; and

FIG. 8 is an illustration for explaining an example of actions of the display supporting function according to the embodiment.

FIG. 9 is a flowchart illustration an example of processing performed by the medical information-processing apparatus.

### DETAILED DESCRIPTION

A medical information-processing system according to an embodiment includes a processing circuitry. The processing circuitry displays medical data in a display region provided in a screen, and performs processing of changing a display region of the medical data while displaying, in the display region, animation showing the process of changing the display range of the medical data according to an operation made with respect to the medical data, or processing of highlighting another medical data related to the medical data subjected to the operation.

Hereinafter, the embodiment of the medical information-processing system will be described, referring to the drawings.

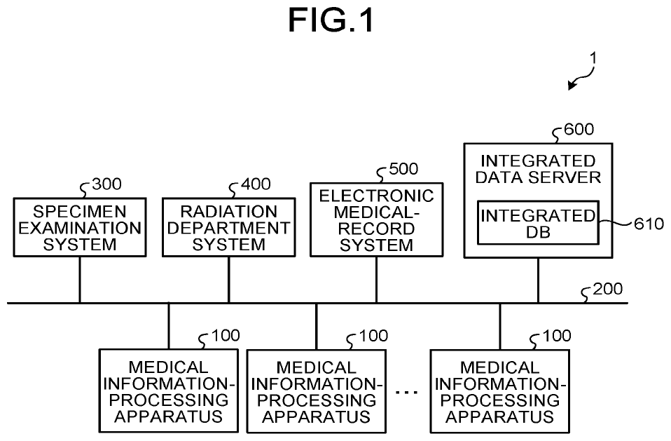

FIG. 1 illustrates a configuration example of the medical information-processing system according to the present embodiment. As illustrated in FIG. 1, a medical information-processing system 1 includes a specimen examination system 300, a radiation department system 400, an electronic medical-record system 500, an integrated data server 600, and a medical information-processing apparatus 100 that are communicably connected to one another through a network 200. For example, the medical information-processing system 1 is installed in a hospital or the like, and the respective systems and the medical information-processing apparatus 100 are connected with one another through the network 200, such as an in-hospital local area network (LAN). Note that the number of the medical information-processing apparatus 100 connected to the network 200 is not particularly limited.

The specimen examination system 300 generates specimen examination data about specimen examination performed with respect to a subject, and stores it in a storage in the system. The specimen examination system 300 transmits the specimen examination data stored in the storage to the integrated data server 600.

The radiation department system 400 stores image data that is acquired by imaging the subject in the storage in the system. Moreover, the radiation department system 400 generates report data about vitals and image examination performed with respect to the subject, and stores it in a storage in the system. For example, the radiation department system 400 includes a picture archiving and communication systems (PACS), and the like. The image examination includes an examination using a computed tomography (CT) image taken by an X-ray CT apparatus, an examination using a magnetic resonance (MR) image taken by a magnetic resonance imaging (MRI) apparatus, an examination using an ultrasound image taken by an ultrasonic diagnostic apparatus, and an examination using an X-ray image taken by an X-ray diagnostic apparatus. The radiation department system 400 transmits the image data and the report data stored in the storage, to the integrated data server 600.

The electronic medical-record system 500 generates medical record data about treatment and care given to the subject, and stores it in a storage in the system. The electronic medical-record system 500 transmits the medical record data stored in the storage in to the integrated data server 600.

The integrated data server 600 is implemented by a computer device, such as a workstation and a server device, that includes a processing circuitry, such as a processor, a storage implemented by a semiconductor memory device, such as a random access memory (RAM) and a flash memory, a hard disk, an optical disk, and the like.

The integrated data server 600 collects various kinds of data from the specimen examination system 300, the radiation department system 400, and the electronic medical-record system 500, and stores it in a storage in the server. Specifically, the storage of the integrated data server 600 holds an integrated database (DB) 610 to store and manage various kinds of data acquired from the radiation department system 400 and the electronic medical-record system 500. Data stored in the integrated DB 610 includes numerical values (measurement values), images, information about health records, and the like, and information indicating a recording time and date of the data, and information for identifying a subject (patient ID), such as a patient.

For example, the integrated DB 610 includes specimen examination data, vital data, image data, report data, medical record data, and the like. The specimen examination data is medical data related to specimen examination acquired from the specimen examination system 300. Moreover, the vital data is medical data related to vitals acquired from the radiation department system 400. Furthermore, the report data and the image data are medical data related to image examination acquired from the radiation department system 400. The medical record data is medical data related to a treatment and care record acquired from the electronic medical-record system 500.

The medical information-processing apparatus 100 acquires various kinds of medical data from the integrated data server 600 through the network 200, and performs various kinds of information processing relating to display of the acquired medical data. For example, the medical information-processing apparatus 100 is implemented by a computer device, such as a workstation, a personal computer, and a tablet terminal.

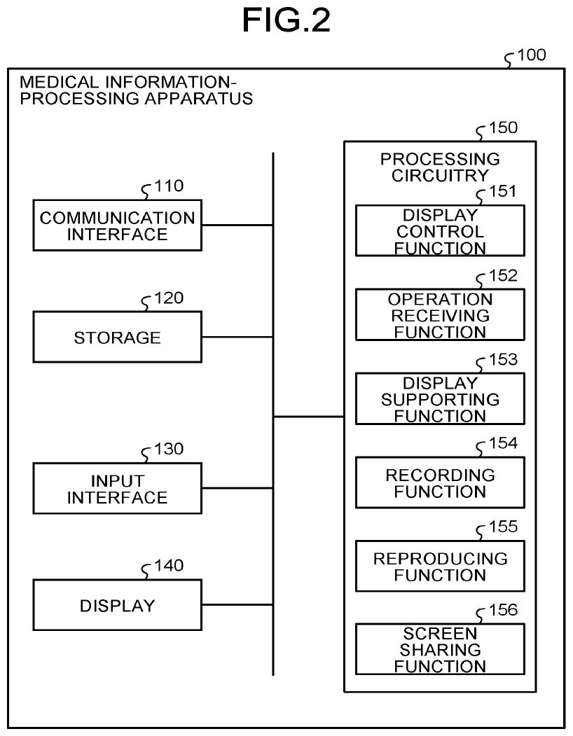

FIG. 2 illustrates an example of a configuration of the medical information-processing apparatus 100. As illustrated in FIG. 2, the medical information-processing apparatus 100 includes a communication interface 110, a storage 120, an input interface 130, a display 140, and a processing circuitry 150.

The communication interface 110 is connected to the processing circuitry 150, and controls transmission and communication of various kinds of data between the medical information-processing apparatus 100 and the respective systems. Specifically, the communication interface 110 receives medical data from the respective systems, and outputs the received medical data to the processing circuitry 150. For example, the communication interface 110 is implemented by a network card, a network adopter, a network interface controller (NIC), or the like.

The storage 120 is connected to the processing circuitry 150, and stores various kinds of data. Specifically, the storage 120 stores medical data received from the respective systems. For example, the storage 120 is implemented by a semiconductor memory device, such as a RAM and a flash memory, a hard disk, an optical disk, and the like. Note that the storage 120 is an implementation example of a storage unit.

The input interface 130 is connected to the processing circuitry 150, and receives an input operation of various kinds of instructions and various kinds of information from an operator. Specifically, the input interface 130 converts the received input operation from the operator into an electrical signal, to output to the processing circuitry 150. For example, the input interface 130 is implemented by a trackball, a switch button, a mouse, a keyboard, a touch pad with which an input operation is made by touching on an operating surface, a touch screen in which a display screen and a touch pad are integrated, a non-contact input circuit using an optical sensor, a voice input circuit, and the like. Note that in the present specification, the input interface 130 is not limited to the one including a physical operating part, such as a mouse and a keyboard. For example, an electrical-signal processing circuit that receives an electrical signal corresponding to an input operation from an external input device provided separately from the apparatus, and that outputs this electrical signal to a control circuit is also included in the input interface 130.

The display 140 is connected to the processing circuitry 150, and displays various kinds of information and various kinds of images. Specifically, the display 140 converts data of various kinds of information and various kinds of images transmitted from the processing circuitry 150 into an electrical signal for display to be output. For example, the display 140 is implemented by a liquid crystal monitor, a cathode ray tube (CRT) monitor, a touch panel, or the like.

The processing circuitry 150 controls the components of the medical information-processing apparatus 100 according to an input operation received from the operator through the input interface 130. Specifically, the processing circuitry 150 stores treatment data output from the communication interface 110 in the storage 120. Moreover, the processing circuitry 150 reads treatment data from the storage 120, and display the data on the display 140. For example, the processing circuitry 150 is implemented by a processor.

The medical information-processing apparatus 100 configured as above is provided for each medical practitioner, such as doctor, nurse, and clinical examination technician, or for each department, and is used for various uses. Specifically, the medical information-processing apparatus 100 is used for creating materials to be used in conference or presentation, and for displaying the materials. For example, a medical practitioner can create a material to explain a condition of the subject based on medical data of the subject, by using the medical information-processing apparatus 100.

Those materials used in a conference and the like described above is generally static medical data in which a particular concerned portion is enlarged or extracted. However, just by presenting static medical data, it is difficult to grasp conditions of periphery of the particular portion or relations with other medical data, and it has not been convenient.

Accordingly, the medical information-processing apparatus 100 according to the present embodiment has a function to improve the convenience of displaying medical data used as a material in a conference as described above.

Specifically, the medical information-processing apparatus 100 includes a display control function 151, an operation receiving function 152, a display supporting function 153, a recording function 154, a reproducing function 155, and a screen sharing function 156. The display control function 151 is an example of a display control unit. Moreover, the operation receiving function 152 is an example of an operation receiving unit. Furthermore, the display supporting function 153 is an example of a display supporting unit. The recording function 154 is an example of a recording unit. Furthermore, the reproducing function 155 is an example of a reproducing unit. Moreover, the screen sharing function 156 is an example of a screen sharing unit.

The display control function 151 displays medical data specified through the input interface 130 on the display 140. Specifically, the display control function 151 acquires medical data of a subject (patient ID) specified through the input interface 130 from the integrated DB 610 of the integrated data server 600, and displays the acquired medical data on the display 140.

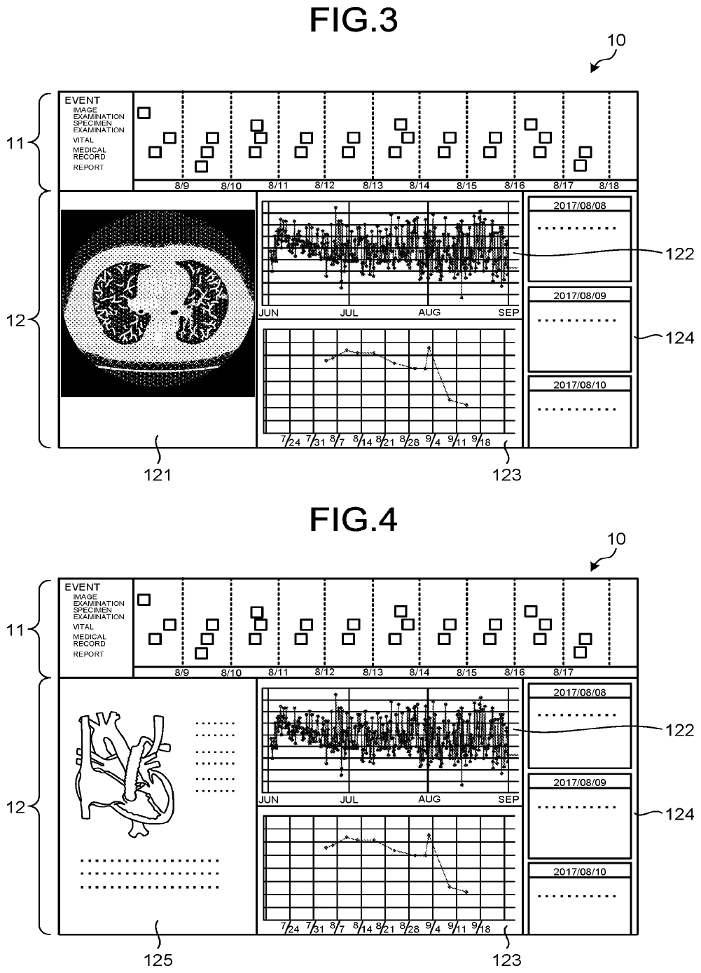

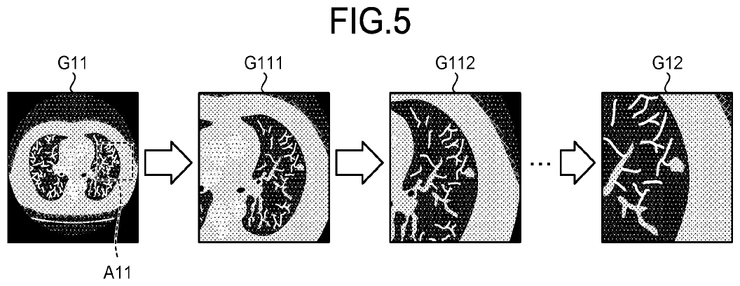

FIG. 3 and FIG. 4 illustrate an example of a screen displayed by the display control function 151. The display control function 151 displays one or more pieces of medical data in the same screen as illustrated in FIG. 3 and FIG. 4 according to a designation by an operator. FIG. 3 and FIG. 4 illustrate a display example of medical data of a particular subject.

As illustrated in FIG. 3, the display control function 151 displays a screen 10 including a timeline display region 11 and a medical-data display region 12 on the display 140.

In the timeline display region 11, events of medical treatment with respect to the subject to be displayed in chronological order. The event means an acquisition event (specimen examination) of specimen examination by the specimen examination system 300, an acquisition event (vitals) of vital data by the radiation department system 400, an acquisition event (image examination, report) of report data and image data by the radiation department system 400, an acquisition event (medical record) of medical record data by the electronic medical-record system 500, and the like. For example, the display control function 151 displays patterns symbolically expressing contents of the events of medical treatment given to the patient, in chronological order.

When receiving an operation to specify a point of time or period from among a display period displayed on the timeline display region 11, the display control function 151 displays contents of medical data for an event performed at the specified point of time or period, in the medical-data display region 12. At this time, the display control function 151 refers to the integrated DB 610 of the integrated data server 600, acquires medical data relating to the event performed at the specified point of time or period, and displays contents of the acquired medical data in the medical-data display region 12.

For example, the display control function 151 refers to image data included in the integrated DB 610, and displays image data relating to image examination performed at the specified point of time or period in an image data display region 121. Moreover, for example, the display control function 151 refers to vital data included in the integrated DB 610, and displays chronological data (graph) showing changes of measurement values relating to vitals taken at the specified point of time or period in a vital-data display region 122. Furthermore, for example, the display control function 151 refers to specimen examination data included in the integrated DB 610, and displays chronological data (graph) showing changes of measurement values relating to specimen examination performed at the specified point of time or period in a specimen-examination-data display region 123. Moreover, for example, the display control function 151 refers to medical record data included in the integrated DB 610, and displays medical record data relating to treatments and cares performed at the specified point of time or period in a medical-record data display region 124.

Layouts and display items in the medical-data display region 12 can be arbitrarily changed. For example, as illustrated in FIG. 4, a report-data display region 125 may be displayed in the medical-data display region 12, in place of the image-data display region 121. In this case, the display control function 151 refers to report data included in the integrated DB 610, and displays report data relating to image examination performed at a specified point of time or period in the report-data display region 125.

Returning back to FIG. 3, the operation receiving function 152 receives an operation to be performed on medical data displayed in the medical-data display region 12 through the input interface 130.

Specifically, the operation receiving function 152 receives an operation to instruct a change of a display range of medical data displayed in the medical-data display region 12. The “display range” herein specifies a range of an image to be actually displayed in the display region, out of the image of the medical data displayed in the display region, such as the image-data display region 121, the vital-data display region 122, and the specimen-examination-data display region 123.

For example, the operation receiving function 152 receives an operation to instruct zoom-in display of a specific region (enlargement operation such as zoom-in operation), an operation to instruct zoom-out of a specific region (reduction operation such as zoom-out operation), an operation to move the display region in any of upward, downward, leftward, and rightward directions (shifting operation), and the like as an operation to instruct a change of display range of the image data to be displayed in the image-data display region 121.

Moreover, for example, the operation receiving function 152 receives an operation to instruct zoom-in display of a specific period (enlargement operation), an operation to instruct zoom-out of a specific period (reduction operation), an operation to move a display range in anterior and posterior directions in time series (shifting operation), and the like as an operation to instruct a change of display range of chronological data to be displayed in the vital-data display region 122 and the specimen-examination-data display region 123.

An operation method to instruct a change of display range is not particularly limited, and various methods an be applied. For example, it may be configured such that by manually specifying a desired range (image region) from image data, a portion of the image data in the specified image region is zoomed-in. Furthermore, it may be configured such that by manually specifying a desired range (period) from chronological data, a portion of the chronological data in the specified period is zoomed-in. Moreover, for example, it may be configured such that a portion or a period operated by a single action, such as one click, may be zoomed-in (or zoomed-out) by setting a fixed enlargement ratio (or reduction ratio) in advance.

Furthermore, the operation receiving function 152 receives a selection instruction of a part of element from medical image displayed in the medical-data display region 12. For example, the operation receiving function 152 receives an operation to select a part of element (for example, a nursing record of a specific date and time) included in the medical record data as an operation with respect to the medical record data displayed in the specimen-examination-data display region 123.

When the above operations are performed with respect to the medical data displayed in the medical-data display region 12, the display supporting function 153 performs display supporting processing according to the operation. Specifically, the display supporting function 153 changes a display method of the medical data subject to operation, and other medical data related to the concerned medical data, according to a type of the medical data subject to the operation and its operation.

For example, when the type of the medical data subject to operation is image data, and the operation is to instruct zoom-in display, the display supporting function 153 controls the display method of the image data subject to operation as follows. First, the display supporting function 153 generates an animation that shows the process of changing a display range from a display range of image data before zoom-in to a display range of the image data after zoom-in. The display supporting function 153 displays the generated animation while switching the display from the display range of the image data before zoom-in to the display range of the image data after zoom-in. That is, the display supporting function 153 changes the display range of image data while displaying the generated animation. The animation is not limited to a moving image, but also includes successive images.

FIG. 5 is an illustration for explaining an example of actions of the display supporting function 153. An image G11 indicates a display range of image data that is displayed in the image-data display region 121 before zoom-in, and an image G12 indicates a display range of the image data after zoom-in.

Suppose that the operation receiving function 152 has received an operation to display a portion of a region A11 in the image G11 in a zoom-in manner. In this case, the display supporting function 153 generates an animation G111, G112, . . . that shows a process of changing from the image G11 to the image G12, and switches the display to the image G12 while displaying the generated animation.

As described, when instructed to change the display range of image data, the display supporting function 153 changes display ranges while displaying a process of change in animation. Thus, a medical practitioner that refers to medical data (image data) can grasp a display range after the change, that is conditions around the region A11, easily by observing a series of process relating the change of the display range.

Generation of the animation G111, G112, . . . may be performed in parallel with switching operation, or may be performed as preprocessing before switching to the image G12. Moreover, the number of pieces of image data (frames) is not particularly limited, but it is preferable to be the number of pieces enabling to smoothly express the process of change of the display range.

Furthermore, for example, when the type of medical data subject to operation is chronological data, and the operation is to instruct zoom-in display, the display supporting function 153 controls the display method of the chronological data subject to operation as follows. First, the display supporting function 153 generates an animation that shows the process of changing a display range from a display range of chronological data before zoom-in to a display range of the chronological data after zoom-in. The display supporting function 153 displays the generated animation while switching the display from the display range of the chronological data before zoom-in to the display range of the chronological data after zoom-in. That is, the display supporting function 153 changes the display range of the chronological data while displaying the generated animation.

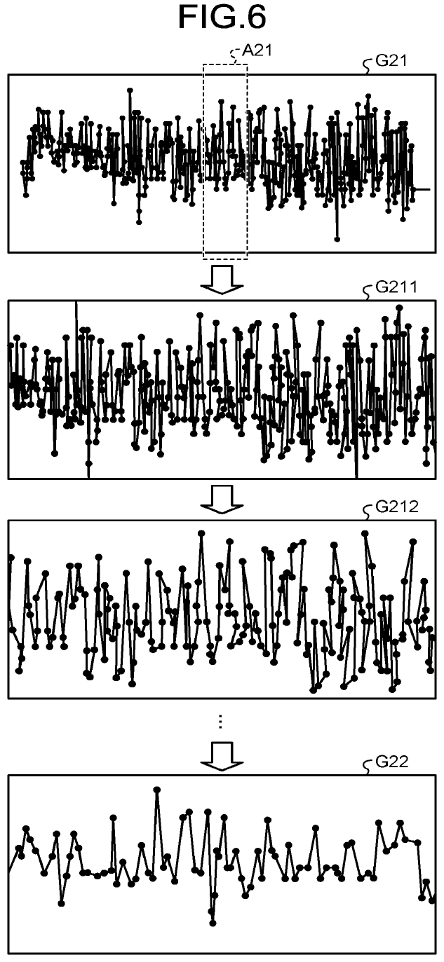

FIG. 6 is an illustration for explaining an example of actions of the display supporting function 153. FIG. 6 is an example of chronological data displayed in the vital-data display region 122. An image G21 indicates a display range of the chronological data before zoom-in in the vital-data display region 122, and an image G22 indicates a display range of the chronological data after zoom-in.

Suppose that the operation receiving function 152 has received an operation to display a portion of a period A21 in the image G21 in a zoom-in manner. In this case, the display supporting function 153 generates an animation G211, G212, . . . that show a process of changing from the image G21 to the image G22, and switches the display to the image G22 while displaying the generated animation.

As described, when instructed to change the display range of chronological data, the display supporting function 153 changes the display range while displaying a process of change in animation. Thus, a medical practitioner that refers to medical data (chronological data) can grasp a display range after the change, that is conditions around the period A21, easily by observing a series of process relating the change of the display range.

With FIG. 5 and FIG. 6, the display method in the case of zoom-in display (enlargement operation) has been described. Similarly, in the case of reduction operation and shifting operation, an animation showing a process of changing a display range is generated, and the change of display range is to be performed while displaying the generated animation.

Moreover, for example, when the type of medical data subject to operation is medical record data including one or more elements, such as character string, and the operation is to instruct selection of a particular element, the display supporting function 153 highlights another medical data relating to the selected element.

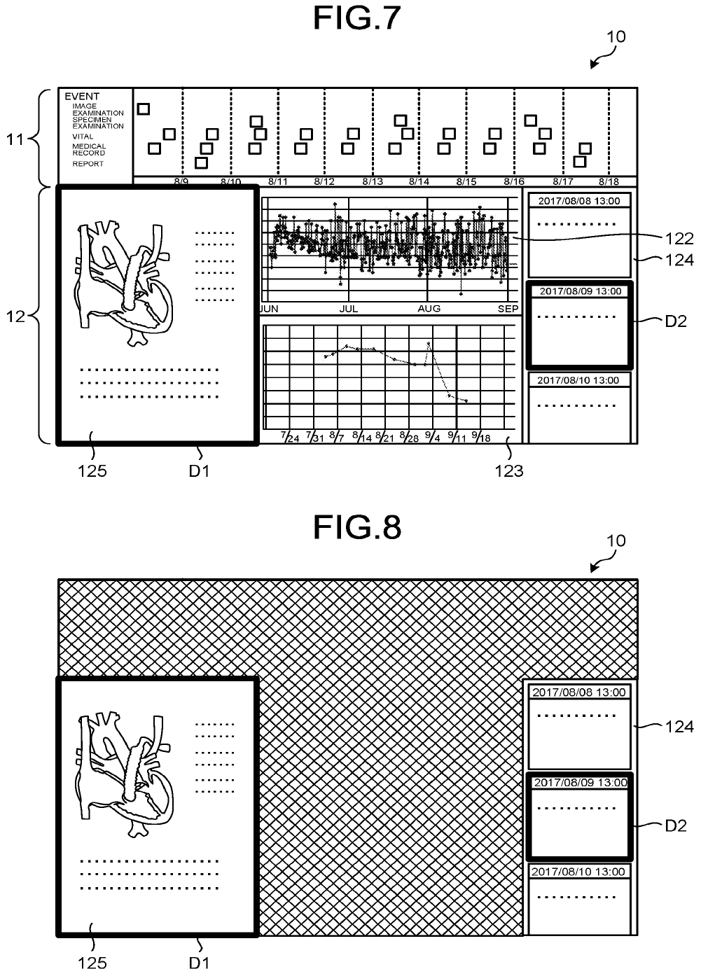

FIG. 7 is an illustration for explaining an example of actions of the display supporting function 153. Note that an screen 10 in FIG. 7 shows a screen similar to one in FIG. 4.

In FIG. 7, for example, when medical record data of a specific date and time of record is selected from among medical record data displayed in the medical-record-data display region 124, the display supporting function 153 sets the date and time of record (date and time) included in the selected medical record data to an element indicating a relation among medical data. Subsequently, based on the set date and time as a reference point, the display supporting function 153 searches for other medical data recorded within a predetermined period (for example, six hours around the time) around the reference point, from among the medical data displayed in the medical-data display region 12. In the medical-record-data display region 124, the other medical data searched for are highlighted.

FIG. 7 illustrates an example in which medical record data of “AUG/09/2017 13:00” is selected from among medical record data displayed in the medical-record-data display region 124, and an example in which medical record data in the report-data display region 125 is highlighted as another medical data relating to the concerned medical record data. Specifically, in FIG. 7, the highlighting is achieved by providing a decoration D1 in a frame shape to the report data.

The method of highlight is not limited to the example in FIG. 7. For example, it may be achieved by flashing the report-data display region 125, or by flashing the decoration D1. Moreover, as a decoration D2 in FIG. 7, the selected medical record data may also be highlighted by a similar method. Furthermore, for example, as illustrated in FIG. 8, it may be configured to highlight only the related medical data by hiding a region other than the selected medical data (report data) and the other medical data (report data) related to the concerned medical data.

As described, when a specific element is selected from medical data, such as medical record data, the display supporting function 153 highlights another medical data related to the selected element. Thus, a medical practitioner can grasp a relation between the selected element and the other medical data easily by observing the highlighted medical data.

In the above, an example in which report data is highlighted has been described, but when a period of the selected element is included in chronological data, such as vital data and specimen examination data, for example, a corresponding period among the chronological data may be highlighted. Furthermore, a corresponding period in the chronological data may be displayed by the method of zoom-in described above.

Moreover, in the example described above, date and time (date and time of record) included in medical data is set to the element indicating a relation among medical data, but the element indicating a relation is not limited thereto. For example, an examination type, a medicine name, and the like may be the element indicating a relation among medical data.

Furthermore, in the above example, search for another related medical data is done from among the medical data displayed in the medical-data display region 12, but it is not limited thereto. For example, medical data of respective events displayed in the timeline display region 11 may be included in a range to be searched.

Returning back to FIG. 3, the recording function 154 records what is performed in the display supporting processing performed with respect to medical data by the display supporting function 153, and thereby generates reproduction data that can reproduce the display supporting processing.

For example, when the display supporting function 153 has performed processing of changing a display range of image data or chronological data in accordance with a change instruction for a display range, the recording function 154 acquires an animation generated by the display supporting function 153, and generates reproduction data that can reproduce the animation. Moreover, the recording function 154 records the generated reproduction data, associating with information enabling to identify medical data subject to operation. Note that it is preferable that the recording function 154 generate a series of reproduction data relating to a change of a display range including a state (image) before and after the change of the display range, not just an animation part.

Furthermore, for example, when the display supporting function 153 has performed processing of highlighting another medical data related to an element of selected medical data, the recording function 154 generates reproduction data including information to identify the other medical data subject to highlighting and a method of highlighting. The recording function 154 records the generated reproduction data, associating with the medical data subject to the selection operation or information enabling to identify the element.

Moreover, the recording function 154 may record a series of operations performed with respect to medical data by an operator in a reproduceable manner by recording reproduction data in order of processing performed by the display supporting function 153. The recorded reproduction data is stored in, for example, the storage 120, or the like.

Note that a subject to be recorded by the recording function 154 is not limited to processing of the display supporting function 153. For example, the recording function 154 may record information enabling to identify medical data displayed in the medical-data display region 12 and its layout, and the like as reproduction data. Furthermore, the medical data to be displayed in the medical-data display region 12 is changed by the display control function 151, processing relating to the change may be recorded as reproduction data.

Moreover, the recording function 154 may be configured to record entire processing performed by the display supporting function 153, or to record a part of the processing. In the latter case, for example, only a change operation for a display range may be a subject to recording, or a subject to recording may be determined according to instruction by an operator. In this case, the recording function 154 may display an operation screen to select whether to record or not on the screen 10. Furthermore, only processing performed with respect to specific medical data may be a subject to recording.

The reproducing function 155 reproduces the reproduction data recorded by the recording function 154. Specifically, when predetermined operation is performed with respect to medical data with which the reproduction data is associated, the reproducing function 155 reproduces the reproduction data associated with the medical data. Thus, the display supporting function performed by the display supporting function 153 with respect to the medical data is reproduced.

For example, when the display supporting processing relating to a change of a display range is recorded as the reproduction data, the reproducing function 155 reproduces the animation generated by the display supporting function 153 by reproducing the reproduction data. Moreover, for example, when the display supporting processing relating to highlighting of medical data is recorded as the reproduction data, the reproducing function 155 reproduces the highlighting of the medical data by reproducing the reproduction data.

The operating method of instructing reproduction of reproduction data is not particularly limited, but because a setting operation, such as specifying an enlargement ratio, is unnecessary, for example, a simple operation, such as one-click operation, may be applied. For example, when a series of operation performed by the operator is recorded, the reproducing function 155 sequentially reproduces the reproduction data each time a click operation is performed.

The reproducing function 155 is assumed to be used at a site, such as a conference, after the entire processing by the display supporting function 153 has been finished, that is after materials have been created by operating medical data by an operator. Therefore, for example, it may be configured to limit a function exclusively, such that either one of the display supporting function 153 and the reproducing function 155 is effective.

By using the display supporting function 153 and the reproducing function 155 in combination, it may be configured such that the display supporting function 153 is used for medical data for which reproduction data has not been recorded, and the reproducing function 155 is used for medical data for which production data has been recorded. Moreover, it may be configured such that an operator displays a material (medical data) at a site, such as a conference while operating the medical data, by using only the display supporting function 153 out of the display supporting function 153 and the reproducing function 155.

The screen sharing function 156 shares a screen by displaying an entire part or a specific part of a screen displayed on the display 140 of the apparatus, on the display 140 of another unit of the medical information-processing apparatus 100 (hereinafter, share destination apparatus) connected through the network. For example, the screen sharing function 156 displays the entire screen displayed on the display 140 of the apparatus of itself on the display 140 of the share destination apparatus operated by a medical practitioner attending the conference or the like.

The screen sharing function 156 share a display effect given by the display supporting function 153 or the reproducing function 155 also, with the screen displayed by the display control function 151. Therefore, the medical practitioner operating the share destination apparatus can view the screen (medical data) in a state displayed on the display 140 of the medical information-processing apparatus 100 operated by the operator that is giving an explanation at the same time.

Moreover, the screen sharing function 156 may be configured to share a display effect displayed on the display 140 of its apparatus and an operation performed with respect to the screen with the share destination apparatus as a part of screen. For example, at a site, such as a conference, even when the same medical data is displayed at each of the multiple medical information-processing apparatuses 100, there is a case in which a layout of a displayed screen varies. In such a case, the screen sharing function 156 may reflect (share) a display effect or a processing (for example, emphasized display, such as highlighting) performed in its own apparatus to medical data same as the medical data being displayed on the display 140 of its own apparatus out of medical data displayed on the display 140 of the share destination apparatus.

Thus, a medical practitioner operating the share destination apparatus can view a screen (medical data) in a state displayed on the display 140 of the medical information-processing apparatus 100 operated by the operator that is giving an explanation, while maintaining the screen layout displayed on the display 140 at the same time.

Note that a screen that the screen sharing function 156 can share with another apparatus is not limited to a screen that is actually displayed on the display 140 of its own apparatus. For example, when a screen (layout, or the like) to be shared, generated in a form, such as reproduction data, is held in a hidden state, the screen sharing function 156 may display the screen to be shared, on the share destination apparatus. Thus, for example, while displaying a screen for conference on the display 140 of its own apparatus, the operator giving an explanation can display a screen (for example, medical data to be a ground for diagnosis) different from the screen. Moreover, in this case, it is preferable that a screen to be shared be selectable.

Furthermore, the screen sharing function 156 may display, when a screen (entire or a part of the screen) to be shared is provided by the screen sharing function 156 of the other medical information-processing apparatus 100, the provided screen on the display 140 of its own apparatus. Thus, the medical information-processing apparatuses 100 can share a screen with one another.

Moreover, when receiving a screen to be shared from the screen sharing function 156 of another unit of the medical information-processing apparatus 100, the screen sharing function 156 may have an operator of its own apparatus confirm whether to display the screen. In this case, for example, upon receiving a screen to be shared from the other unit of the medical information-processing apparatus 100, the screen sharing function 156 displays a message to confirm whether to display it and operators on the display 140 of its own apparatus. Subsequently, when instructed to display the screen the by the operator, the screen sharing function 156 displays the screen to be shared, provided by the other unit of the medical information-processing apparatus 100 on the display 140 of its own apparatus. Furthermore, as another example, the operator sets setting information indicating whether to display a shared screen in advance, and the screen sharing function 156 may display the shared screen provided by the other unit of the medical information-processing apparatus 100. This enables to prevent a shared screen provided by another unit of the medical information-processing apparatus 100 from being displayed at the share destination apparatus without intention and, therefore, improves the convenience.

It is preferable to be configured such that an original screen is displayed also when a shared screen provided by the other unit of the medical information-processing apparatus 100 is displayed at its own apparatus. For example, the screen sharing function 156 may display the shared screen provided by the screen sharing function 156 of the other unit of the medical information-processing apparatus 100 and the original screen in a switchable manner with tabs or the like. Furthermore, for example the screen sharing function 156 may store contents (layout and the like) of the original screen when displaying the shared screen provided by the screen sharing function 156 of the other unit of the medical information-processing apparatus 100, and may display the shared screen and the original screen in a switchable manner according to an instruction from the operator.

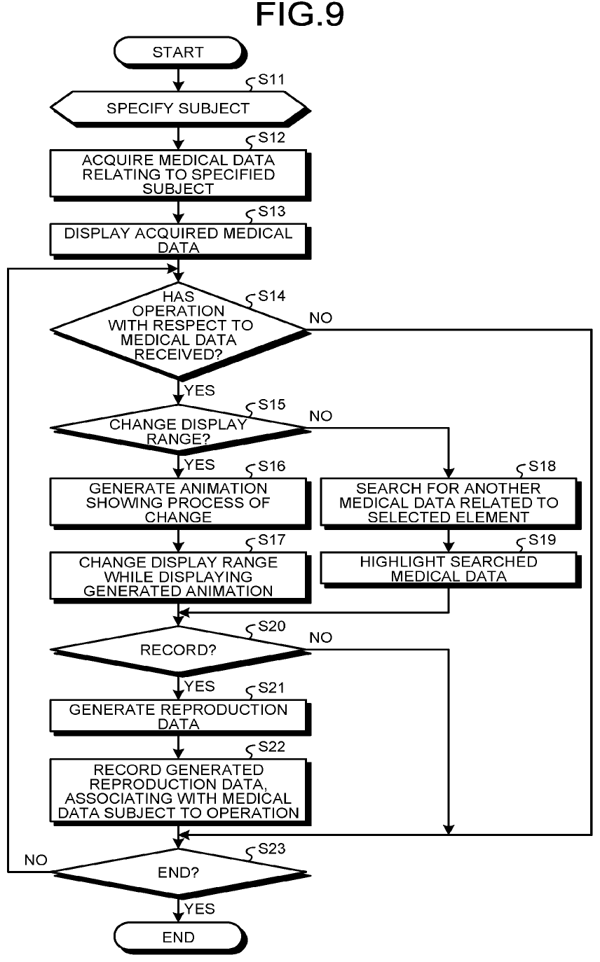

Next, actions of the medical information-processing apparatus 100 described above will be described, referring to FIG. 9. FIG. 9 is a flowchart illustrating an example of processing performed by the medical information-processing apparatus 100.

First, a subject to be displayed is specified by an operator by a patient ID input through the input interface 130, or the like (step S11). When a subject is specified, the display control function 151 acquires medical data relating to the subject from the integrated DB 610 (step S12). Subsequently, the display control function 151 displays the acquired medical data in the same screen as illustrated in FIG. 3 (step S13).

Subsequently, the operation receiving function 152 determines whether an operation with respect to the medical data has been received (step S14). When an operation with respect to the medical data has not been received (step S14: NO), the operation receiving function 152 shifts the processing to step S23.

Moreover, when the operation receiving function 152 has received an operation with respect to the medical data (step S14: YES), the display supporting function 153 determines whether the operation is to instruct a change of display range of the medical data (image data or chronological data) (step S15).

When the operation is to change a display range (step S15: YES), the display supporting function 153 generate an animation showing a process of changing a display range (step S16), and changes the display range while displaying the generated animation (step S17). The display supporting function 153 then shifts the processing to step S20.

On the other hand, when the operation is to select an element in the medical data (step S15: NO), the display supporting function 153 searches for another medical data related to the selected element (step S18). Subsequently, the display supporting function 153 displays the searched medical data in a highlighted manner (step S19). The display supporting function 153 shifts the processing to step S20. When no medical data related to the selected element is present, the display supporting function 153 returns the processing to step S14.

Subsequently, the recording function 154 determines whether to record the processing performed by the display supporting function 153 (step S20). For example, when it is set to record only processing related to a change of display range, the recording function 154 determines to record the processing on condition that it has been determined that a change of display range is instructed at step S15. Moreover, for example, when instructed to record it by an operator, the recording function 154 determines to record it.

Having determined to record it (step S20: YES), the recording function 154 generates reproduction data based on the processing performed by the display supporting function 153 (step S21). Subsequently, the recording function 154 records the generated reproduction data, associating with information enabling to identify medical data subject to operation (step S22), and shifts to step S23. Moreover, having determined not to record it (step S20: NO), the recording function 154 directly shifts to step S23. Note that the recording function 154 records reproduction data in order of processing performed by the display supporting function 153.

Subsequently, the operation receiving function 152 determines whether it has been instructed to end the main processing (step S23). When it has not been instructed to end it (step S23: NO), the operation receiving function 152 returns the processing to step S14. When it is determined to end the main processing (step S23: YES), the operation receiving function 152 co-operates with the display control function 151, to end the main processing by closing a screen displaying medical data, or the like.

In the processing of FIG. 9, it is determined whether to record reproduction data each time the display supporting function 153 performs processing, but it is not limited thereto. For example, it may be configured to record data automatically without performing determination. Furthermore, it may be configured to determine whether to record data right before the end of the processing in FIG. 9.

As described above, in the present embodiment one or more pieces of medical data are displayed in the same screen, an operation with respect to the medical data is received, and a display method of the medical data or another medical data related to the medical data is changed according to characteristics of data and what is performed in the operation. Thus, a medical practitioner attending a conference can grasp conditions of periphery of a particular portion focused by a person that has created a material, and a relation among medical data easily by referring to a screen displayed on the display 140 of the medical information-processing apparatus 100. Therefore, it is possible to improve convenience of displaying medical data.

The embodiment described above may be appropriately modified to be implemented by changing a part of the configuration or the functions included in the medical information-processing apparatus 100. In the following, some modifications according to the above embodiment will be described. In the following, points that differ from the above embodiment will be mainly explained, and detailed explanation of points in common with what has been described already will be omitted. Moreover, the modifications described hereafter may be performed independently, or may be performed in combination.

### First Modification

While the medical information-processing apparatus 100 is configured to include the display control function 151, the operation receiving function 152, the display supporting function 153, the recording function 154, the reproducing function 155, and the screen sharing function 156 in the embodiment described above, it may be configured such that a part of or all of these functions are provided in an apparatus other than the medical information-processing apparatus 100.

For example, it may be configured such that the integrated data server 600 provides a part of or all of the respective functions described above to the medical information-processing apparatus 100 in a form of web service or the like. Moreover, in this case, the integrated data server 600 is not limited to one computer device, but may be implemented by multiple computer devices connected through a network.

In the embodiments described above, an example in which a display control unit, an operation receiving unit, a display supporting unit, a recording unit, a reproducing unit, and a screen sharing unit are implemented by the display control function 151, the operation receiving function 152, the display supporting function 153, the recording function 154, the reproducing function 155, and the screen sharing function 156 of the processing circuitry 150, respectively has been described, but embodiments are not limited thereto. For example, the display control unit, the operation receiving unit, the display supporting unit, the recording unit, the reproducing unit, and the screen sharing unit in the present application may be implemented by a form of implementing similar functions by hardware, or a combination of hardware and software, in addition to implementation by the display control function 151, the operation receiving function 152, the display supporting function 153, the recording function 154, the reproducing function 155, and the screen sharing function 156 described in the embodiment.

Furthermore, the term “processor” used in the above description signifies, for example, a circuitry such as a central processing unit (CPU), a graphics processing unit (GPU), an application specific integrated circuit (ASIC), a programmable logic device (for example, a simple programmable logic device (SPLD), and a complex programmable logic device (CPLD)), and a field programmable gate array (FPGA). The processor reads and executes a program stored in the storage 120, and thereby implements a function. It may be configured to install the programs directly in a circuitry in the processor, instead of storing the programs in the storage 120. In this case, the processor implements a function by reading and executing a program installed in the circuitry. Moreover, the processor of the present embodiment is not limited to be configure as a single circuit, but may be configured with multiple independent circuits integrated into one processor, to implement the function.

Moreover, a program to be executed by the processor is stored in a storage, such as a ROM to be provided. This program may be recorded in a computer-readable recording medium, such as a compact disk read-only memory (CD-ROM), a flexible disk (FD), a compact disk recordable (CD-R), and a digital versatile disk (DVD), in a file data of an installable format or an executable format, to be provided. Furthermore, this program may be stored in a computer connected to a network, such as the Internet, and be provided by being downloaded through the network. For example, this program has a module structure including each of the functions described above. As actual hardware, the CPU reads and executes the program from the storage device, such as a ROM, and the respective modules are thereby loaded on the main storage device to be generated thereon.

According to at least one of the embodiments described above, convenience of displaying medical data can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information-processing system comprising
a processing circuitry that displays medical data in a display region provided in a screen, and that performs, according to an operation with respect to the medical data, any one of processing of changing a display range of the medical data while displaying an animation that shows a process of changing the display range of the medical data in the display region, and processing of displaying another medical data related to the medical data subject to the operation in a highlighted manner.

2. The medical information-processing system according to claim 1, wherein
when receiving an operation to zoom-in or zoom-out the display range of the medical data, the processing circuitry displays an animation that shows a process from a display range before zoom-in or zoom-out to a display range after zoom-in or zoom-out in the display region.

3. The medical information-processing system according to claim 1, wherein
when an operation to shift a display range of the medical data is received, the processing circuitry displays an animation that shows a process from a display region before shift to a display range after the shift in the display region.

4. The medical information-processing system according to claim 1, wherein
the processing circuitry displays respective pieces of the medical data of different data types in different display regions provided in the identical screen.

5. The medical information-processing system according to claim 1, wherein
when an operation to select a specific element included in the medical data is received, the processing circuitry displays medical data related to the specific element among medical data other than the medical data subject to the operation in a highlighted manner.

6. The medical information-processing system according to claim 1, wherein
the processing circuitry displays the medical data subject to operation in a highlighted manner.

7. The medical information-processing system according to claim 1, wherein
the processing circuitry hides data except the medical data subject to operation and another medical data related to the medical data.

8. The medical information-processing system according to claim 1, wherein
the processing circuitry further records reproduction data that enables to reproduce processing performed with respect to the medical data according to an operation, associating with the medical data subject to operation, and reproduces the reproduction data recorded in an associated manner with the medical data, according to an operation with respect to the medical data.

9. The medical information-processing system according to claim 1, wherein
the processing circuitry shares a display content of the screen with a plurality of apparatuses connected to a network.
